(19) United States
(12) Patent Application Publication
(10) Pub. No.: US 2006/0246497 A1
(43) Pub. Date: Nov. 2, 2006

(54) Ultra-rapid DNA sequencing method with nano-transistors array based devices

(75) Inventors: Jung-Tang Huang, Taipei (TW); Cheng-Hung Tsai, Taipei (TW)

Correspondence Address: Jung-Tang Huang, 5F., No.7, Lane 10, Sec. 2, Bade Rd., Da-an District, Taipei City, 106, TW

(73) Assignee: Jung-Tang Huang

(21) Appl. No.: 11/409,793

(22) Filed: Apr. 25, 2006

(30) Foreign Application Priority Data
Apr. 27, 2005 (TW) 094113562

(51) Int. Cl. C12Q 1/68; G06F 19/00; C12M 1/34

(52) U.S. Cl. 435/6; 435/287.2; 702/20; 977/702; 977/924

(57) ABSTRACT

The invention disclosed a method based on nano-transistors for ultra-rapid DNA sequencing. The method provides micro-fabricated electrodes, which are applied for stretching and driving DNA in the solution to overpass the fabricated carbon nanotube transistors (CNTFETs) array. When DNA molecules are moved perpendicularly to the axial direction of carbon nanotubes, the DNA molecule will touch carbon nanotube surface base after base such that it can measure current flow varied from different base according to charge transfer between the DNA molecule and the nanotubes. Due to this charge-transferred mechanism, the invention could achieve DNA sequencing and make a record or compare with the precedent in the database of DNA molecules.

### BACKGROUND

1. Field of Invention

This invention relates to a method of and apparatus for ultra-rapid nucleic acid sequencing. More particularly, this invention relates to methods and apparatus with which base sequences of different length of nucleic acid molecules in the solution can be determined ultra-rapidly and automatically by devices of stretching and driving and by the nano-transistors of detecting.

2. Description of Related Art

Nucleic acid molecule includes DNA, RNA and etc. It needs first to extract and purify the nucleic acid molecule from the cell nucleus before the analysis. The procedure is: 1. Soak the cells into the detergent or use other physical methods to lyse the cell membrane. 2. Remove the debris and protein enzyme by centrifugal methods and leave only the nucleic acid molecules in the solution.

The analysis for nucleic acid can be divided into two processes: the first one is diagnosis which determines whether the existence of specific base pairs or not; the second process is sequencing, which detects the real sequence of the base pairs. There are five basic chemical procedures for general analysis, for example referring to C. H. Mastrangelo, M. A. Burns, and D. T. Burke “Microfabricated Devices for Genetic Diagnostics,” Proceedings of the IEEE, Vol. 86, No. 80, 1998, Aug.

1. Chemical amplification: Heat the double-stranded fragments to separate them into two single-stranded segments and then utilize the enzyme polymerase to synthesize single-stranded into double-stranded fragments. This procedure is called polymerase chain reaction (PCR). The typical amplifying procedure is using the high-temperature resistant Taq polymerase enzyme which extracted from a heat-resistant microorganism, Thermos aquaticus; and mixed the unknown template of the nucleic acid with enough amounts of nucleotides (dNTP's) and primers of the determined duplicate starting point.

2. Add fluorescent dyes in the fragments of the nucleic acid molecules.

3. Restriction enzyme was used for fragmentation or digestion.

4. Separation: In general, electrophoresis and capillary electrophoresis are often used. Both of them are used for separation of different length of the nucleic acid fragments.

5. Sequence reading: Two common schemes are described as follows.

A. Sanger sequencing scheme: Add a small amount of ddNTP's (ddA, ddC, ddG, or ddT) to make the polymerization incomplete and stop at the ddNTP's. Then apply the capillary electrophoresis and use fluorescent microscope or other optical schemes for readings.

B. The other scheme: Use the fixed nucleic acid molecule probe array to facilitate the hybridization procedure.

The above-mentioned schemes use many dose of expensive enzyme and specimens. The PCR or the making of the nucleic acid molecule probe is also complicated and expensive.

Recently, there is some rapid DNA sequencing methods disclosed. The U.S. Pat. No. 6,093,571, which used the electrophoresis to drive the nucleic acid to pass through the nanopore and measured the ion in the solution to find the influence on the amount of the nucleic acid molecule bases blocked when passing through the nanopore; then detected the base is which of the AGCT. However the possible difficulties are: how to control the thermal noise and increase the signal to noise ratio (SNR) under the room temperature, in order for measuring base pairs; how to efficiently make the curly nucleic acid to overpass nanopore and how to make the length of the nanopore is smaller than 0.5 nm, the diameter is around 2 nm to improve the accuracy to one base pair. However these will all increase the difficulties during manufacturing and will have a high possibility of blocking the channel, making it hard to clean up. In addition, if we put channel protein, such as maltoporin (LamB) pore, onto biochemical thin film as the nanopore; it will cause problems of the interaction between DNA and channel protein. Also the length of the biochemical channel is too long to efficiently identify the base pairs. Further more, there are some drawbacks for making it into several channels.

In conclusion, we need an invention: it is able to read the DNA sequences and is easy to combine with the reading circuit; moreover, the size of the device for reading the nucleic acid molecules should be close to the interval of the nucleic acid molecule bases and can be easily made by using the semiconductor manufacture process or microelectromechanical system (MEMS) techniques. It also needs an integrated system for controlling the nucleic acid molecules and applying electric fields to stretch nucleic acid molecules before entering the reading device. Therefore the bases are able to pass through the channel one by one to make the sequence of the nucleic acid molecule be clearly identified. The measurement process should be implemented under room temperature with high SNR.

### SUMMARY

In one aspect, the invention relates to a method of ultra-rapid sequencing for nucleic acid molecule. The invention disclosed a method based on nano-transistors, making the speed for sequencing faster than any other present technology.

In yet another aspect, the invention could reduce cost because it does not need to use PCR, gel electrophoresis or any other procedures which requires biochemical materials.

In another aspect, the invention relates to a method of ultra-rapid sequencing for longer nucleic acid molecule, unlike the prior arts of employing hybridization, which can only perform well in case of using shorter nucleic acid probes.

In still another aspect, the invention provides low-cost and effective methods to stretch the nucleic acid molecule before entering the channel. The sequence for the nucleic acid can be clearly identified because the bases come to contact with the nanotube one by one.

In a further aspect, the invention relates to a method that the fabricated carbon nanotube transistor (CNTFET) is designed in an array. Therefore it is a method that can sequence multi-fragments of the nucleic acid molecules at the same time.

In another aspect, the invention provides an integrated chip, which is made by using the semiconductor manufacture processes or MicroElectromachnical System (MEMS) processes.

In yet another aspect, the invention relates to a method of ultra-rapid sequencing for nucleic acid molecule from a variety of biological source. It can also be applied for sequencing the nucleic acid molecule with the original molecular length.

In a further aspect, the invention features an apparatus for performing ultra-rapid sequencing. The apparatus includes several channels. Each channel provides two or more carbon nanotube transistors in a tandem arrangement for the purpose of sequencing the same nucleic acid molecule. The sequence data attained from these two or more carbon nanotube transistors can be cross-compared and corrected by the bio-information technology to increase the accuracy.

These and other features and advantages of the present invention will be presented in more detail in the following specification of the invention and the accompanying figures, which illustrate, by way of example, the principles of the invention.

### BRIEF DESCRIPTION OF DRAWINGS

The invention may best be understood by reference to the following description taken in conjunction with the accompanying drawings that illustrate specific embodiments of the present invention.

FIG. 1A is a schematic drawing of nano-transistor DNA sequencing apparatus, wherein the transistor is a p-type transistor with the nanowire of p-type and Si substrate of n-type; and FIG. 1B is a schematic drawing of nano-transistor DNA sequencing apparatus, wherein the transistor is a n-type transistor with n-type nanowire and p-type Si substrate.

FIG. 2 is a complete flow process of DNA sequencing that may be used with the present invention.

FIG. 3A is a schematic drawing of DNA stretching and driving method that may be used with the present invention; FIG. 3B is the schematic drawing of DNA stretching method that may be used with the present invention; and FIG. 3C is the schematic drawing of the array-type DNA sequencing apparatus with self-comparison function that may be used with the present invention.

FIG. 4A is a method in according with the present invention to drive and stretch the single DNA molecule into the cavity area; and FIG. 4B is a method to drive the single DNA molecule to the nano-transistor reading area.

FIG. 5 is a circuit in according with the present invention, including driving, controlling and measuring modules

FIG. 6 is a detailed circuit drawing of the I-V (shunt-shunt) feedback transimpedance amplifier (TIA) that may be used with the present invention.

FIG. 7A is the output voltage wave form while the 500 nA, 10 MHz sine wave current is applied on the TIA chip; and FIG. 7B is the output voltage wave form while the 25 nA, 10 MHz sine wave current is applied on the TIA chip.

FIG 8 is an embodiment of nano-transistor that may be used with the present invention.

FIG. 9 is a schematic drawing of the nanotube-transistor for DNA sequencing in according with the present invention.

FIG. 10 is an AFM image of the CNTFET that Mo is the electrode material.

### DETAILED DESCRIPTION

Reference will now be made in detail to some specific embodiments of the present invention including the best modes contemplated by the inventor for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

In the following description, numerous specific details are set forth in order to provide a thorough understanding of the present invention. The present invention may be practiced without some or all of these specific details. In other instances, well known process operations have not been described in detail in order not to unnecessarily obscure the present invention.

Furthermore, techniques and mechanisms of the present invention will sometimes be described in singular form for clarity. However, it should be noted that some embodiments can include multiple iterations of a technique or multiple applications of a mechanism unless noted otherwise.

The system and method of the present invention will be described in connection with DNA sequencing. However, the system and method may also be used for RNA sequencing. Additionally, the system and method may be used for other genetic analysis of DNA or RNA.

This invention disclosed a method based on nano-transistors for ultra-rapid DNA molecule sequencing. Fernando Patolsky disclosed a method of nanowire channel for nano-transistor to detect single virus in 2004 (Ref. Fernando Patolsky, Gengfeng Zheng, et al. “Electrical detection of single viruses.” PNAS, Vol. 101, No. 39, pp. 14017-14022, 2004, September). The nano-transistor is an extremely sensitive real-time virus detector. When a virus under test, which has surface positive charge, travels through the solution and comes in contact with the antibody on the nanowire. The conductance is decreasing because the depletion of p-type nanowire. On the other hand, when the virus under test has negative charge on its surface and comes in contact with the antibody on the p-type nanowire, the conductance is rising. The virus travels through the solution to pass through the surface of the nanowire. However the DNA molecules in the solution are wound around like a filamentous ball shape so it is unable to stretch the DNA molecules in the solution to pass through the surface of the nanowire. Therefore the invention disclosed a method of using dielectrophoresis and electrophoresis to control the DNA molecules and make them to be stretched to pass through the surface of nanowire in order for the measuring, and recording the increase or decrease of the electric current between the source and drain as every base contact with the nanowire. Thus the purpose of the nucleic acid sequencing can be achieved.

Further more, the size of the virus is normally around 100 nm and the diameter for the nanowire is around 10 nm. Comparing with the nucleic acid molecule base the nanowire is still too big. This invention disclosed a method of using nanowire with diameter less than 1 nm such as single-walled nanotube to make nano-transistor. This invented design of the p-type and n-type nanowires employ heavy-dpoed substrates as back gate and functionalize the nucleic acid molecule slightly to make its electric conduction increase. (Ref. Kei Shimotani, Taishi Shigematsu et al. “An advanced electric probing system: Measuring DNA derivatives.” JOURNAL OF CHEMICAL PHYSICS VOLUME 118, NUMBER 17, 2003) In addition, the polymers can also be used to cover on the nanotube to cause it to be more sensitive to molecular hydrogen. (Ref. Jing Kong, Michael G. Chapline, and Hongjie Dai. “Functionalized Carbon Nanotubes for Molecular Hydrogen Sensors”. Adv. Mater. 2001, 13, No. 18, September 14) Both functionalization methods are for the efficiency of nucleic acid sequencing.

Nucleic Acid Molecule Sequencing Mechanism.

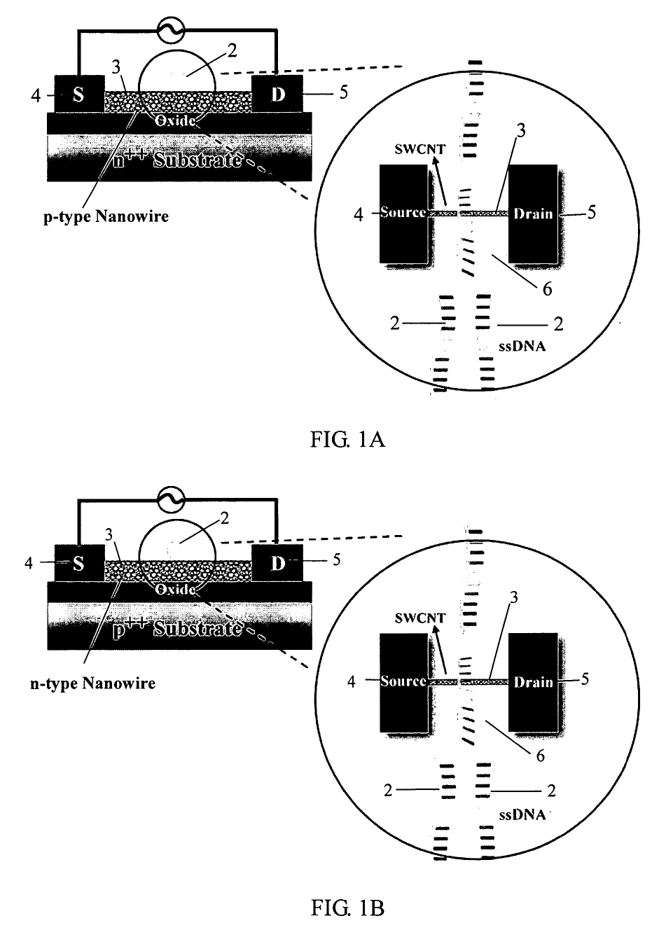

This invention also disclosed an array-type sequencing device for DNA molecule. Each unit in the array is called the channel. FIG. 1A and B is the array channel: the reading device 1 for DNA molecule. The main part of the device is the carbon nanotube transistor (CNTFET) and it is using the micro-fabrication technology to make a p-type or n-type CNTFETs 3. The deposit metal on the two sides above the conducting wire were source/drain 4/5. It uses dielectrophoresis and electrophoresis to stretch the single-stranded DNA molecule 2 and guide it through the channel 6 formed by the photo resist between the electrodes; eventually causing it to come in contact with the tube wall of the carbon nanotube. Because the different bases of single-stranded DNA molecule (ssDNA) have different chemical molecules therefore when the DNA molecule passes through the nanotube, the positive and negative electric charge will cause transfer with the carriers of the nanotube to make the conductance decrease or increase. The nanowire is the nano-channel for the field effect transistor and it is exposed to the exterior substance, therefore it is very sensitive with the electron and the hole transfer even a slightly deviation can be detected. By this way the invention makes sequencing of the nucleic acid molecule possible. The reading target for the invention is the DNA or message RNA (mRNA), because the DNA includes genes and its intergenic regions. The mRNA is exactly a part of the gene code so the sequencing of mRNA should be also important.

The main concepts for nucleic acid molecule sequencing are:

1. Four bases A, G, C, and T of the DNA can cause obviously different carrier changes on the surface of nanotube of nano-transistor such that they are identified respectively with neglected influence from the size variation of the micro-channel.

2. Be sure that the DNA molecule is lead by 5′ to pass through the channel and it is not blocked when passing through the channel.

3. The speed for the DNA molecule when passing through the channel should not be affected by the length or combination of the nucleic acid molecules. The time taken for each base to pass through the nanotube is similar.

4. The multiple channels are for the reading and sequencing of the nucleic acid molecule at the same time. The multiple channels also increase the speed for the sequencing regardless of the types or length of the nucleic acid molecules.

5. The results of sequence made by the same channel can be compared with each other to eliminate the mistake.

An Embodiment Strategy

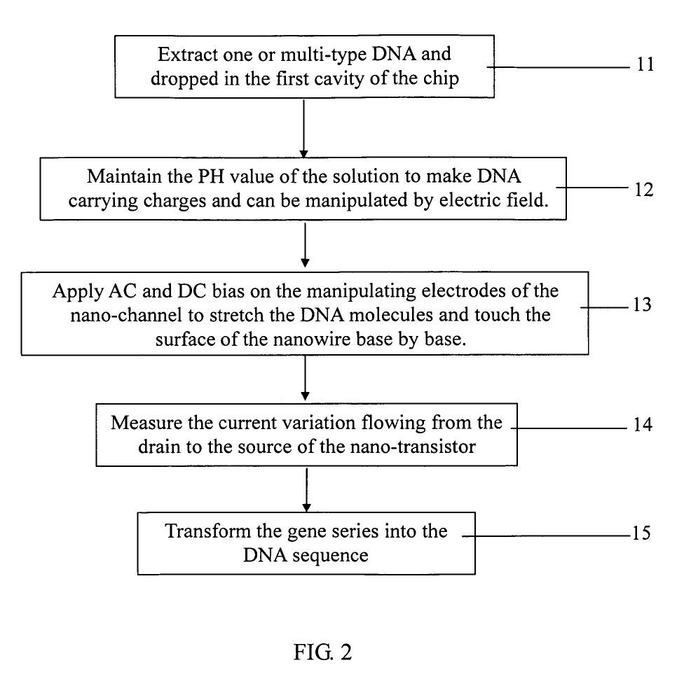

The sequencing procedure for nucleic acid molecule is shown in FIG. 2, and will be described as follows.

Procedure 1 11: Drop solution containing one or multiple kinds of DNA molecules into the apparatus.

Use typical methods to extract the one or multiple kinds of DNA molecules, which are to be sequenced will be placed into the exact consistency of chloride salt solution.

Procedure 2 12: Maintain the single-stranded status for DNA molecule or messenger ribonucleic acid (mRNA).

Increase the heat to 70˜95° C. to maintain the single-stranded status and no secondary structure for DNA molecule or mRNA. The single-stranded nucleic acid molecule will carry negative electric charges and the adjoin bases will be mutually exclusive as long as the PH level is around 7˜8. The single-stranded DNA or mRNA will also maintain the nearly straight status.

Procedure 3 13: Straighten the nucleic acid molecule in order to pass over and touch the nanowire base by base.

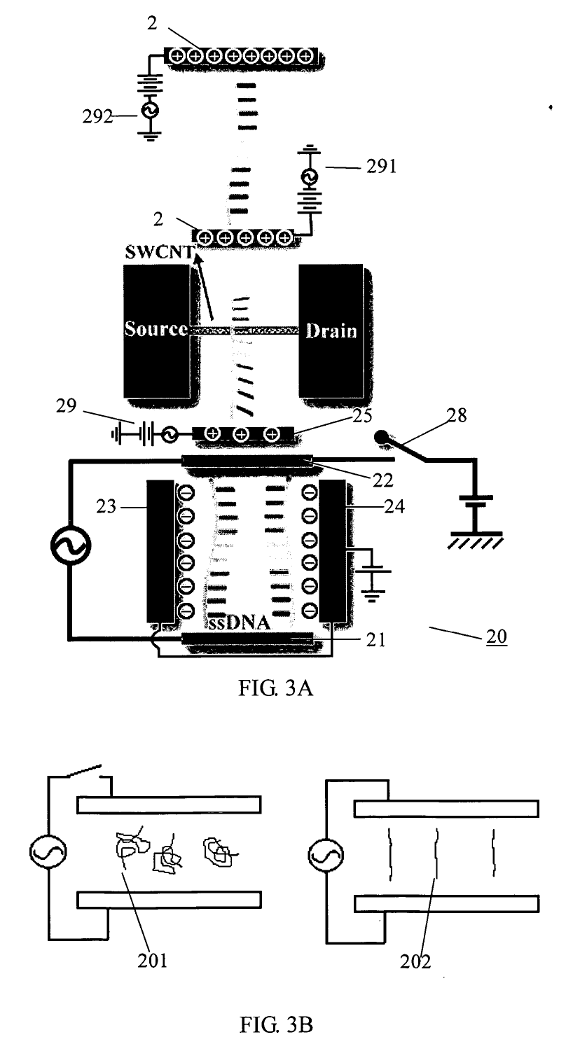

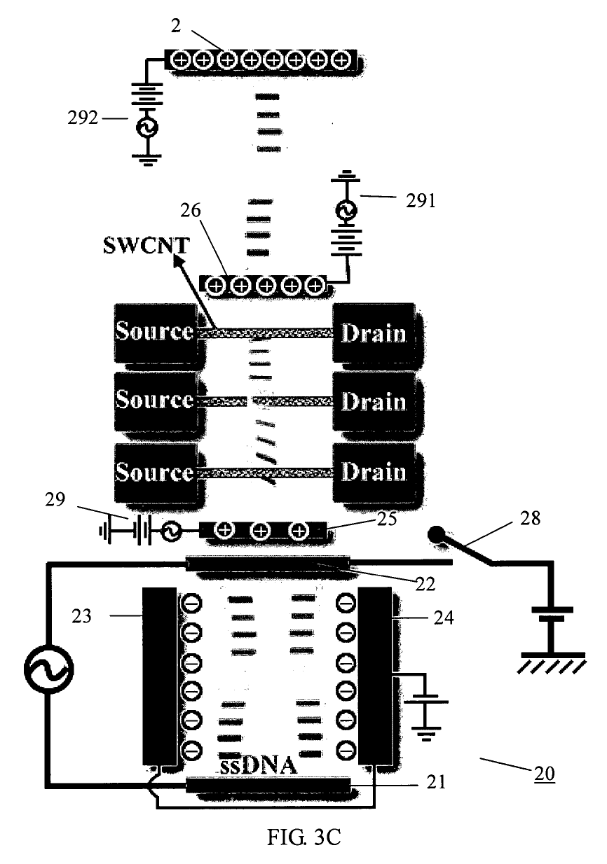

Referring to FIG 3A, the electrode system 20 includes the evaporated electrodes 21, 22, 23, 24, 25, 26. Provide solution with the nucleic acid molecule between the two electrodes 21, 22. The nucleic acid molecule 201 is rolled up. The nucleic acid molecule 202 is straightened, when applying an electric field between the two electrodes. Referring to FIG. 3B (S. Suzuki, et. al., “Quantitative Analysis of DNA Orientation in Stationary AC Electric Fields Using Fluorescence Anisotropy,” IEEE Trans. Industry Applications Vol. 34, NO. 1, January/February, 1998, pp 75-83), this invention installs a DC electrode/signal input wire 28 at the electrode 22 and then uses the DC bias voltage to attract the nucleic acid molecule which has negative electric charges to move toward the second electrode 22. Thus the straightened nucleic acid molecule can continue being pulled toward the direction with bigger electric field. The invention also employs a set of negative DC bias voltage electrodes 23, 24 beside the electrodes 21 and 22. The nucleic acid molecule will be expelled into middle region and not have any chance to roll up because the nucleic acid molecule carries negative electric charges in the solution under the exertion of negative DC bias voltage. In order to maintain the nucleic acid molecule straightened status and make it move toward the bigger electric field, a DC, AC electrode/signal wire 29 is set at electrode 25. By adding DC bias voltage at electrode 25 to make field strength greater than that of the AC bias voltage signal 28 therefore the straightened nucleic acid molecule can continue moving forward. In addition, DC bias on the back gate of the nano-transistor could be negative to result in inducing positive charges distributed on the nanowire and the surface of the dielectric on the back gate such that the approaching DNA molecule is attracted to contact the nanowire.

A DC/AC signal input wire 291 is connected to the electrode 26. Apply DC bias voltage to the electrode 26 with amplitude larger than the DC bias voltage 29 applied to the electrodes of the upstream station and if exert an AC signal timely to confirm the DNA molecule being stretched straightly and pass through the area upon the top wall of the carbon nanotubes (CNTs). When the DNA carrying with negative charges touches the surface of the CNTs with positive charges, the invention apparatus will read the signal variation of the transistor properties while the bases pass through and it would be used to sequence DNA. Finally, connect a DC/AC signal wire 292 and apply a DC bias voltage and AC signal timely on the electrode 27 and it will be confirmed that the DNA molecules will be stretched and kept moving. The DC bias voltage would be bigger than that applied on the electrode 291 and the DNA molecule would approach to field with higher electric charge and keep moving forward. The DNA sequencing process would be finished while the DNA molecule pulled out the transistor.

Procedure 4 14 Measuring the Conductance of the Nanowire

There is only one stretched DNA molecule pass and touch the CNT's surface and every base of the DNA molecules could be stayed on the nanowire for a short period. The electron or hole would be transferred on the nanowire while the base of the DNA is contacting with the nanowire. The transferring of carriers could be measured by the current/voltage transimpedance circuit.

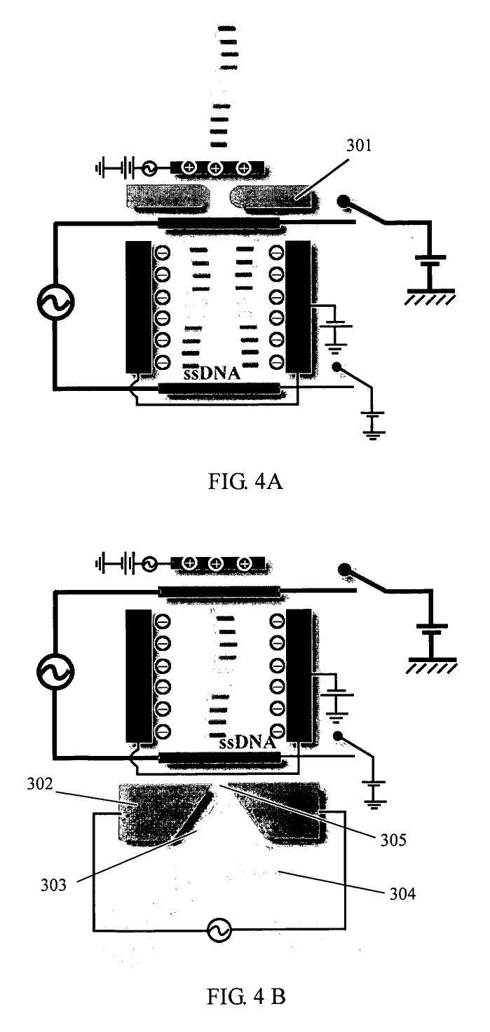

As shown in FIG. 4A, a nanopore 301 with diameter 2-4 nm could be set in downstream of the first cavity 303. The DNA molecules in the solution are guided to pass through the nanopore by the electrophoresis and dielectrophoresis force. (Keisuke MORISHIMA et al., “Manipulation of DNA Molecule Utilizing the Conformational Transition in the Higher Order Structure of DNA,” Proceedings of the 1997 IEEE International Conference on Robotics and Automation Albuquerque, pp. 1454-1460, 1997.) Because there is only one DNA molecule can pass through the nanopore, the device will guarantee only one DNA molecule enter the sequencing area of the nano-transistor at one time.

In the FIG. 4B, there could be two non-parallel electrodes 302 in front of the first cavity 303. The two neighboring electrodes are convergent toward the first cavity. Apply electrophoresis and dielectrophoresis force in the solution to guide the rolled DNA 304 to pass through the shrinking hole 305. Because the space of the nano-hole is small enough to let only one DNA molecule to pass through the channel, it would be confirmed that only one DNA molecule would be driven into the nano-transistor reading area at one time.

Procedure 5 15 DNA Sequencing

Generally, the bases of the DNA molecule will transfer electrons with the free carriers of the CNTs. Because the diameter of the CNTs is about 1 nm, it is easy to recognize the slightly difference and the GATC sequence will be recognized apparently (FIG. 9). Use the multi-bits A/D converter to transform the GATC sequence and store the sequence that would be used for some purpose in the future in the memory of the microcontroller. In order to debug the DNA sequence caught from the same channel, the present invention arranges three CNTFETs in the same channel in tandem (FIG. 3C) to read the same DNA molecule. After reading the DNA sequencing, the Bioinformatics technology would be used to debug the sequencing data that read by the three CNTFETs and to raise the accurate rate of the sequencing.

Circuit Theory of CNTFETs

The current in the channel of CNTFETs is about several pA to hundreds of nA. In order to minimize the size of the current measuring circuit and integrate it on a single chip, the circuit is designed in the way of integrated circuit in this invention. The explanations of the driving controllers and measurement modules are as follows:

Refer to FIG. 5, the driving controllers and measurement modules 30 can be divided into 5 independent sub-circuits, including:

1. I/V converter; 2. DC-voltage; 3. AC-voltage; 4. High speed A/D converter; 5. Microcontroller.

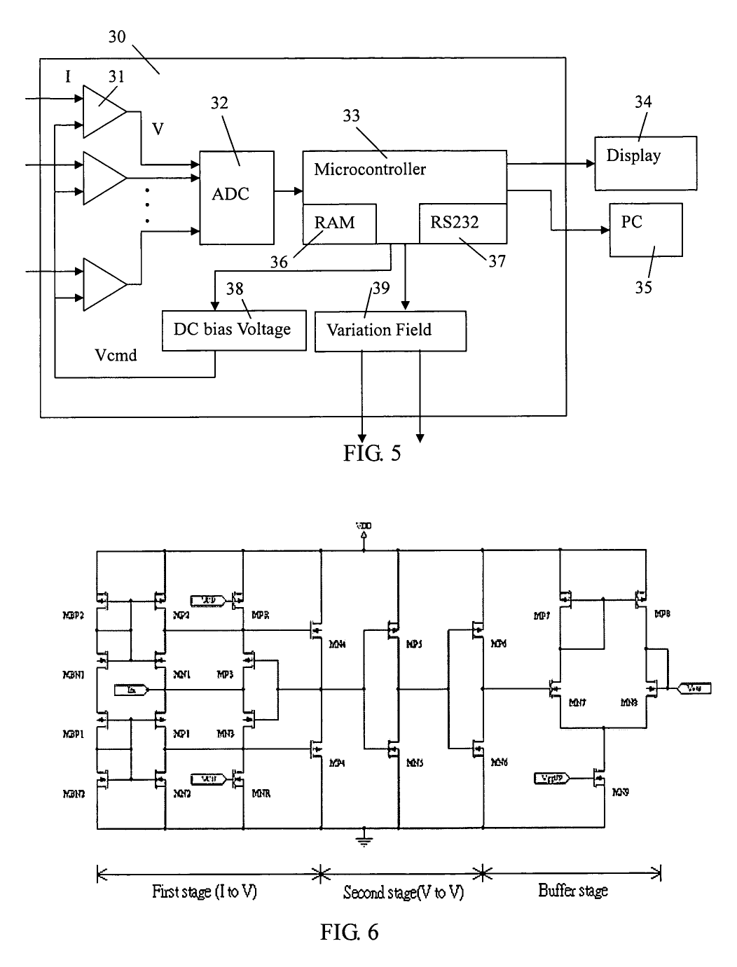

I/V converter 31, the purpose is to transfer the ultra small current in CNT-Transistor to several mV. Here we make use of trans-impedance amplifier to complete this part, and the detail will be explained later with FIG. 6.

Multi-bits and high speed A/D converter 32, the purpose is to digitize the six possible voltage levels into multiple bits. The six possible levels include a reference voltage, a non-touching voltage occurred as no base contact the CNT, and four different voltage levels corresponding to the four bases AGCT contacting the CNT surface. The basic architecture of the ADC is flash circuit (reference: D. A. Johns and K. Martin, 1997, Analog Integrated Circuit Design, pp. 507-513), the values of leveling resistances are regulated by the difference of voltages changed for whether contacted with the 4 bases or not. The speed of translation is 10-200 MHz, and it can read multi-channels or multi-transistors by way of a multiplexer.

AC-voltage 39, the purpose is to add 1 MHz high electrical field between two electrodes to stretch the DNA molecules.

DC-voltage 38, the purpose is to cooperate with AC-voltage to stretch the DNA molecules, which means DC-voltage can be added between electrode 21 and electrode 22, electrode 23 and electrode 24. In order to minimize the effect of noise, we provide a way to lower down the speed of DNA molecule passing through the shell wall of CNT, and this will make each base stay temporarily on the nano-conducting wire. Basically, the operation period of DC-voltage with 10-150 mV will be controlled in 1-10 μs.

Microcontroller 33, its function includes: regulate the voltage level and frequency of AC-voltage, voltage level of DC-voltage, conducting time and non-conducting time of synchronous signals, the connection of high speed A/D converter, the translation and storage of genetic sequencing data, and comparison of known genetic sequence stored in memory 36, and showing results on LCD displayer 34. In other way, it can also transfer genetic sequencing data through RS-232 communication protocol 37 to save in a personal computer 35. Microcontroller can also be taken place of off-the-shelf products, and the built-in memory 36 can also be expanded by additional memories. Therefore, it will enlarge the number of sequencing channels and elevate the length of DNA molecule in every channel in this invention.

As far as the current measurement of transistor is concerned, referring to FIG. 6, it can use a current amplifier with CMOS process to make the circuit of current amplifier on a single chip, and combine CNT-transistor with flip-flop as a system on a chip.

Referring to FIG. 5, we cascade the circuit stage by stage with DC coupling, totally 3 stages. The first stage is I/V conversion. The second stage is V/V conversion, in order to get higher gain. The third stage is buffer stage. In practical situation of circuit implementation, the input impedance of input stage is mainly affected by the trans-conductance of MN3 and MP3 (g_{mN3} & g_{P3}) on the route of feedback. Therefore, in order to increase the testing flexibility, the triode-operated-MOSFET MNR (MPR) is connected between the GND (VDD) and the source of MN3 (MP3), which functions as a small resistance. The linear-region turn-on resistance of both MNR and MPR can be adjusted via controlling V_{CN} and V_{CP}, respectively. For a good linearity, the control voltage V_{CN} and V_{CP} should be tuned simultaneously to keep the balanced operation of the feedback devices. (reference: Ping-Hsing Lu, Chung-Yu Wu, and Ming-Kai Tsai,” Design Techniques for Tunable Transresistance-C VHF Bandpass Filters”, IEEE Journal of Solid-State Circuits, Vol. 29 Issue: 9, pp. 1058-1067 September 1994.)

The following experiments were implemented to prove the ability of trans-impedance amplifier:

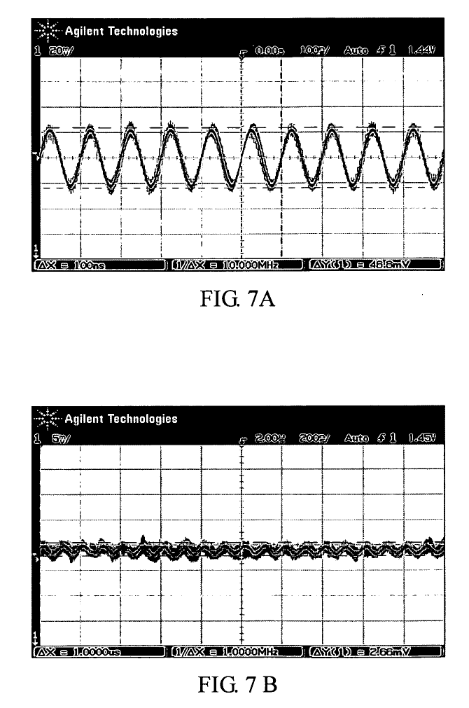

Giving power supplies and bias voltages to the chip, the output will have a dc-voltage about 1.44V. Then providing a sin-wave voltage signal Vin generated from a function generator, after going through a precise resistance of 1MΩ, the signal can be equivalent to a sin-wave current signal Iin≅Vin/1MΩ Ampere. It can get a sin-wave voltage about 1.44V of dc-voltage level at output port. By tuning Vin, decrease the input signal step by step and observe the output signal to analyze and conclude the characteristics of circuit. First, input a sin-wave voltage signal with 500 mV, 100 MHz, and it can be equivalent to a sin-wave current signal with 500 mV, 100 MHz. Observing the output signal from oscilloscope as shown in FIG. 7A, the current signal is about 25 nA, and the measurement result is shown in FIG. 7B. The result of a trans-impedance is more than 90 dB, and it is good enough to be used in this object. It also could use other TIA circuit which would be easy to do by the experienced circuit designer. Here would not go into details.

Apply a suitable electric signal on the source to drain of the CNTFET. In order to avoid the electrochemical reaction on the CNT's surface while the large DC bias voltage is applying on, it could be applied −10 mV voltage on the drain of the p-type nano-transistor (apply positive 10 mV while the nano-transistor is n-type nano-transistor) and connect the source to the ground. It would be measured the μA signal from the source end to the drain end. (Ref. Robert J. Chen, Sarunya Bangsaruntip et al. “Noncovalent functionalization of carbon nanotubes for highly specific electronic biosensors.” 4984-4989 PNAS Apr. 29, 2003 vol. 100 no. 9.); It also could be applied a 30 mV, 200 Hz˜80 Hz AC signal between the source and drain to be the driving voltage of the transistor and put a CMOS current amplifier chip used to measure the nA level signal. Record the variation of the current while DNA molecule pass through and transform into the DNA sequence.

At the gate side, the back gate of a p-type nano-transistor is conducted with a minus bias voltage, which is adjustable under different circumstances. The voltage is generally set between 0V to −5V. On the contrary, an n-type nano-transistor is subjected to a plus voltage instead, and it is also in the range from 0V to 5V. Too large voltage exertion should be avoided, or it may cause the isolation layer, SiO₂, to be knocked through by electronic holes and electrons, making the transistor damaged.

Steps of the Nano-Transistor Fabrication

The steps for fabricating the nano-transistor based on the micro-manufacturing techniques as following:

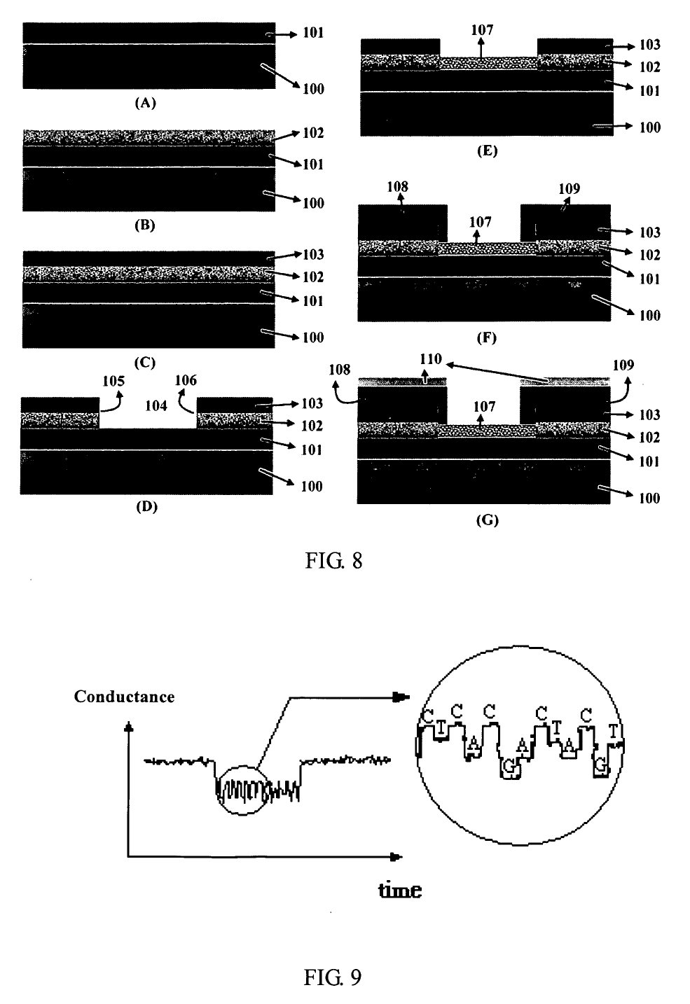

Step 1: Referring to FIG. 8A, a n-type (100) silicon is initially selected to serve as the first substrate 100 and then deposit a layer of SiO₂ film 101 on it with the thickness about 50 nm-100 nm using LPCVD.

Step 2: Referring to FIG. 8B, coating the mixture of oxide and catalyst 102 on the SiO₂ layer 101 utilizes SOG (Spin-on-glass) progress. The catalyst is mainly made up by a series of substances of TEOS (tetraethyl orthosilicate), alcohol, and catalyst ions (Fe, Co, Ni) respectively. A two-step heating method is then adopted. To begin with, put the layer coated with the catalyst under the heating environment for one hour with 100 to 120° C. Secondly, it is also kept for one hour period but with far higher temperature between 350 to 500° C. for the benefit of prompting the joining ability between both the first isolation layer 101 and the second catalyst 102 layer as well.

Step 3: Referring to FIG. 8C, use the same SOG coating process as the next step to coat another layer of oxide 103, e.g. TEOS (tetraethyl orthosilicate) solution, and in the wake of the next step is soft bake drying.

Step 4: Referring to FIG. 8D, combine photolithography with etching processes to etch a hole 104 on layers 102 and 103, piercing down to the topside of the SiO₂ isolation layer 101. Let the sidewalls 105 on the catalyst layer 102 and the sidewalls 106 on the second SiO₂ layer be exposed.

Step 5: Referring to FIG. 8D, the nanotube 107 approximate to 1 nm could be successfully grown up from the sidewall 105 in the CVD chamber set at 850° C., for the reactant source, alcohol, is admitted into the catalyst layer 102 in advance. According to the result above mentioned, the diameter of the nanotube on the sidewall must be less than 0.7 nm, meaning the distance approximates to 0.34 nm between two adjoining bases of the DNA.

Step 6: Referring to FIG. 8F, this invention provide a process to coat a layer of photoresist on the nanowire to define the desired patterns of the electrode and then deposit Pd on it. Afterwards, by using a common lift-off process to let the both ends of Source 108 and Drain 109 contacted with the nanowire. The metal deposited also contains the stretched electrodes 21, 22, 23, 24 of nucleic acid molecule. Too thick electrodes are unnecessary for the driving electrodes 25, 26, 27 of nucleic acid molecule. As a result, this invention makes use of the similar processes of photolithography, etching and lift-off again to finish with the driving electrodes 25, 26, 27 of nucleic acid molecule.

Step 7: Referring to FIG. 8G, the general nanowires may have p-type characteristic. This invention, however, directly uses p-type nanowire as channel of the transistor and n-type substrate to act as the gate. Likewise, the nanowire initially doped to become n-type channel together with p-type substrate as the gate is also practicable. (Ref. Ali Javey, Ryan Tu et al. “High Performance N-Type Carbon Nanotube Field-Effect Transistors with Chemically Doped Contacts.” NANO LETTERS Vol. 5, No. 2 345-348, 2005.)

### EXAMPLE OF EMBODIMENT

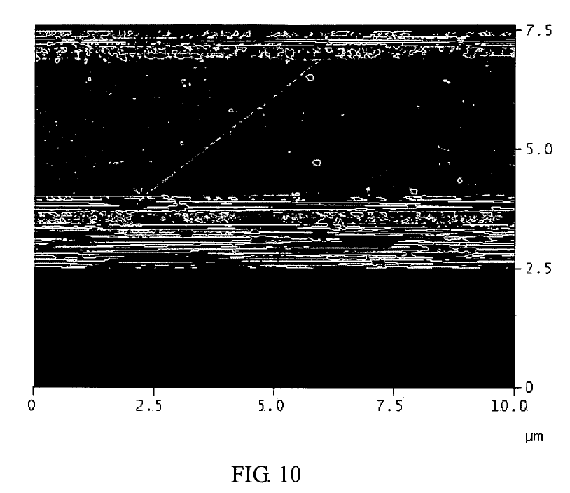

The CNTs synthesis by CVD methods have diameters in the range of 1˜2 nm, noble metals such as Mo or Pd are used as Source and Drain of the CNTFETs, the distance between Source and Drain are varied from 1˜5 μm which can be changed depends on different demands. The AFM image of a CNTFET using CVD methods is shown in FIG. 10. The successful catalysts and growth conditions controlling can synthesize semiconducting SWCNTs with high throughput, and also can bridge two electrodes efficiently, and on-off ratio is higher than 10⁴ as long as the fine quality isolation layer is paved under the nanotubes and free from current leakage at all.

Further, a micro-fluidic channel between Source and Drain is made by E-Beam lithography or standard photolithography technology, this fluidic channel can efficiently manipulate DNA in the middle of the CNTFET, and effectively control DNA molecules touch CNT surface base after base, the current flow variation from different base according to charge transfer between the DNA molecule and CNTs can be detected, consequently, the ultra-rapid DNA sequencing can be realized.

Either a CMOS chip with capability for nano-ampere measuring combined with a CNTFET by flip-chip technology or a method of aligning CNTs from CNT-contained solution on a CMOS chip by Dielectrophoresis (DEP) can connect the signal between a CNTFET and CMOS, both of them leading to accurate signal measurement and record the condition of carrier through CNTs efficiently, the in situ detection and measurement can lower the noise and record the signal variation reliably. FIG. 9 shows a schematic diagram of recorded signals with different DNA bases by using a CNTFET.

In order to avoid the situation of more than one base touched the CNT surface simultaneously resulting in single base is unidentifiable; the CNTs with smaller diameter must be used. In the CVD method, controlling the concentration and size of catalysts and reducing the growth periods can efficiently synthesize CNTs with small diameter; even down to 0.3 nm can be synthesized. In the DEP method, using the smallest CNTs for aligning also can fabricate CNTFET with small diameter CNTs. Under these controls, we can predict the CNT-walled width to down to 0.34 nm or below and this dimension just matches the space of between each base ˜0.34 nm, consequently, DNA molecules can touch CNT surface base after base with high efficiency.

Due to the symmetry of electric structures of double-strand DNA, there are two reversed direction can be observed after applying a bias. The single-strand DNA, however, with no electric symmetry, which can be manipulated easily. We only need to make high electric charge on 5′ head, by utilizing the alternating current field combined with DC bias we can firstly control the 5′ head pass through the CNT surface; besides, recording the DNA sequences with high reliability can be achieved by introducing Bioinformatics' technology.

In conclusion, this invention combined with integrated circuit processes and micro-electro-mechanical system (MEMS) technologies to fabricate CNTFETs or nanowire field-effect transistors (NWFETs). This invention utilizes these nanoelectronics devices to implement DNA sequencing and assures it can reach ultra-fast and high-reliable DNA sequencing.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An apparatus for rapid DNA sequencing, including at least one channel on a substrate, wherein each channel comprising:
a first cavity which can be dropped solution containing the single-stranded DNA molecules;
at least one nano-transistor comprising:
a semiconducting nanowire such as single-walled carbon nanotube;
a drain and source electrodes that said nanowire bridges on and can be
applied an AC voltage or low DC voltage;
and a back gate for the nano-transistor that can be applied a DC bias;
at least two pairs of electrodes perpendicular to the direction of DNA molecules transportation, set in front and back of the nano-transistor to be applied AC fields to stretch the DNA molecules before and after entering into the nano-transistor, and also can be applied positive DC bias on the electrodes to let the stretched DNA molecules arrive and pass the nano-transistor from said first cavity;
a set of measuring circuit connected to the nano-transistor that could measure the current variation while the bases pass through and contact with the nanowire;
a controller that could be applied for:
controlling the sign and magnitude of the DC bias, the amplitude and frequency of the AC fields;
conditioning the measured current signal of the nano-transistor into base sequences; and
recording or comparing said base sequences with the precedent in the database of DNA molecules.

2. The apparatus in claim 1 wherein said DC bias on the back gate of the nano-transistor could be negative to result in inducing positive charges distributed on the nanowire and the surface of the dielectric on the back gate such that the approaching DNA molecule is attracted to contact the nanowire.

3. The apparatus in claim 1 wherein said positive DC bias applied a larger amplitude of voltage on latter pair of electrodes used for stretching the DNA molecules, consequently, the DNA molecules with negative charges are attracted to the downstream of the channel through the nano-transistor, then the stretched DNA molecules can touch the surface of the nano-transistor.

4. The apparatus in claim 1 wherein said nano-transistor and measurement circuit could be installed more than two sets in a tandem arrangement such that the same DNA molecules could be sequenced several times, and the sequence data read by more than two nano-transistors would be cross-compared by the Bioinformatics technology to raise the accurate rate of the sequencing.

5. The apparatus in claim 1, further comprising DC bias electrodes which could be set on the right and left side of the DC stretching electrodes to confine the DNA molecules with negative charges in the middle area of the channel and be stretched straight easily.

6. The apparatus in claim 1, further comprising nanopore with 2-4 nm diameter which could be set in front or back of said first cavity so that the DNA molecule would be guided to pass through the nanopore by the electrophoresis and dielectrophoresis force in the solution, thereby only one DNA molecule would be driven into the nano-transistor measuring area at one time.

7. The apparatus in claim 1, further comprising two nonparallel electrodes could be set in front of and converge to said first cavity so that electrophoresis and dielectrophoresis force can be applied to guide the DNA to let only one DNA molecule to pass through the nanopore and be driven into the nano-transistor reading area at one time.

8. The apparatus in claim 1 wherein said nanowire has diameter less than 0.7 nm.

9. A method for rapid DNA sequencing with multi-channel is to apply multi-channel DNA sequencing apparatus containing at least one nano-transistor with a nano-wire, at least one set electrodes applied DC bias voltage, one set current measurement circuit and one controller, the method comprising:
(a) Dropping ion solution containing one or multiple kinds of DNA molecules on said channel;
(b) Applying DC/AC electric field on the electrodes to let the DNA molecules be stretched straight, driven through and contact with the nanowire of the nano-transistor;
(c) Measuring the current variation of the nano-transistor while the bases of DNA molecules pass through and contact the nanowire; and
(d) Conditioning and converting the measured current signal into the base sequences, which thereby being recorded or compared with the DNA molecule database further.
